(19) United States
(12) Patent Application Publication
(10) Pub. No.: US 2011/0130709 A1
(43) Pub. Date: Jun. 2, 2011

(54) SYSTEMS AND METHODS FOR ELECTRIC FIELD ASSISTED DELIVERY OF THERAPEUTIC AGENTS

(76) Inventor: Colleen Stack N'diaye, Durham, NC (US)

(21) Appl. No.: 12/790,766

(22) Filed: May 28, 2010

Related U.S. Application Data
(60) Provisional application No. 61/182,200, filed on May 29, 2009.

(51) Int. Cl. A61N 1/30

(52) U.S. Cl. 604/21

(57) ABSTRACT

Methods of iontophoretically delivering therapeutic agents to diseased tissue, such as for tumor therapy, are disclosed. In one embodiment, the distal end of an endoscopic device is transorally introducing into the gastro-intestinal tract. A delivery element is advanced from the endoscopic device through a wall of the gastro-intestinal tract and into an adjacent pancreas. A therapeutic agent is delivered from the delivery element into the pancreas, an electric field is generated to drive the emitted therapeutic agent into surrounding tissue of the pancreas, causing the emitted agent to penetrate the surrounding tissue.

This application claims the benefit of U.S. Provisional Application No. 61/182,200, filed May 29, 2009, which is incorporated herein by reference.

INVENTOR Colleen Stack N'diaye

### BACKGROUND

The delivery of therapeutic substances to poorly vascularized tissues is a significant unmet need. In particular, solid tumors, such as those developed in pancreatic cancer, are frustrating to oncologists because of the modest delivery of drugs to the tumor. Pancreatic solid tumors are characterized by a low level of vascularity and a high hydrostatic pressure which makes it difficult for chemotherapy treatments delivered via parenteral routes to accumulate in the tumor either by convective transport or diffusion. In addition, with conventional drug delivery, high concentrations of drug in the tumor can only be obtained with systemic drug levels that produce substantial systemic toxicity. Unfortunately, to achieve uniform drug penetration within the tumor, systemic administration relies heavily on a well-developed and homogenous vascular network which is not available in solid tumors as stated above. Drug/radiation therapy combinations are also used extensively in the clinic, but the toxicity from systemic delivery is a major impediment when only local radiation sensitization is needed.

Iontophoretic transport is a technique in which ionic substances are driven into tissue of interest using an electric field. Transdermal iontophoresis has been successfully used to externally deliver various therapeutics across the relatively avascular and impenetrable barrier of the stratum corneum. Some prior applications describe devices for use in iontophoretic delivery of therapeutics within the body. See, for example, EP 0 438 078, entitled IONTOPHORESIS DEVICE and EP 1 925 335, entitled CATHETER TYPE IONTOPHORESIS CATHETER, and U.S. Pat. No. 4,411,648 entitled IONTOPHORETIC CATHETER DEVICE. However, the use of drug delivery to internal locations driven by an electric field has not been adequately explored.

The present application describes catheters suitable for iontophoretic delivery of therapeutic substances.

### BRIEF DESCRIPTION OF THE DRAWINGS

FIG. 1 schematically illustrates positioning of a disclosed system for use in delivering therapy to pancreatic tumors.

FIG. 2 is a side elevation view of a distal portion of a first embodiment of an electric field assisted delivery system.

FIG. 3 is similar to FIG. 2 but shows an alternative system.

FIG. 4 is a perspective view showing an alternative to the catheter shown in FIGS. 2 and 3.

FIGS. 5A and 5B are similar to FIG. 4 and show yet another alternative catheter. FIG. 5C is a distal end view of still another alternative catheter.

FIGS. 6A through 8 illustrate an alternative system for electric field assisted delivery of therapeutic agents, in which:

FIG. 6A schematically illustrates introduction of a guidewire into the body;

FIG. 6B illustrates the catheter system, with the anchor in the expanded position;

FIGS. 7 and 8 illustrate dilators suitable for use over the guidewire of the system.

### DETAILED DESCRIPTION

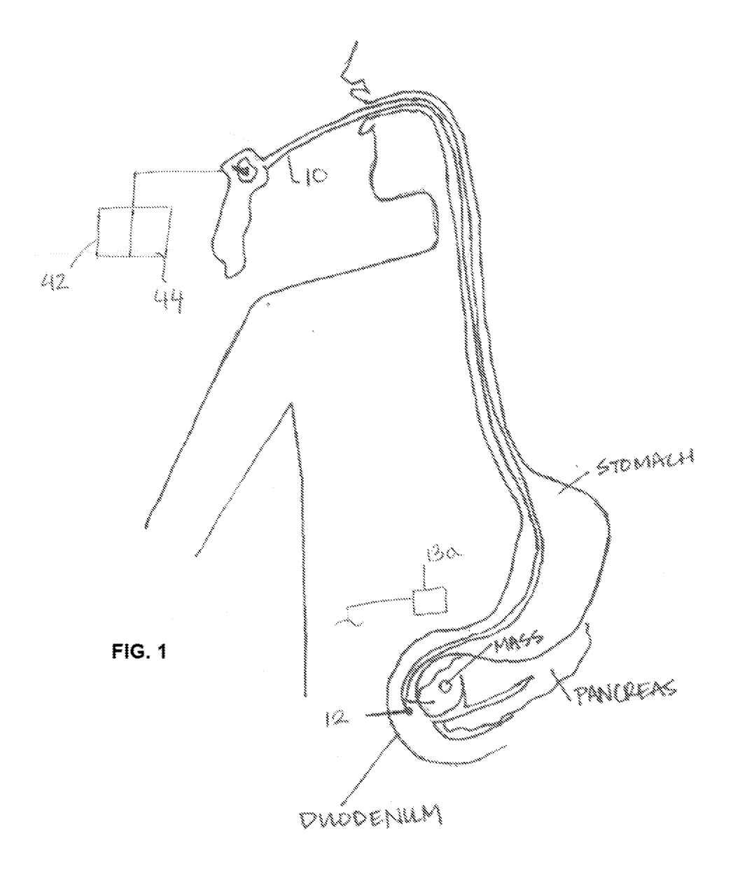

FIG. 1 illustrates an endoscopic approach for delivering substances, electrical therapy or iontophoretic therapy to pancreatic tumors. The illustrated embodiment employs an endoscopic access device 10 which may be a multi-channel endoscope. An ultrasound probe 12 is positioned at the distal end of the access device 10 and may be integral with the access device or a separate ultrasound catheter inserted through the access device 10. In one embodiment, the access device and ultrasound probe may be similar to those used for endoscopic ultrasound guided fine needle aspiration procedures.

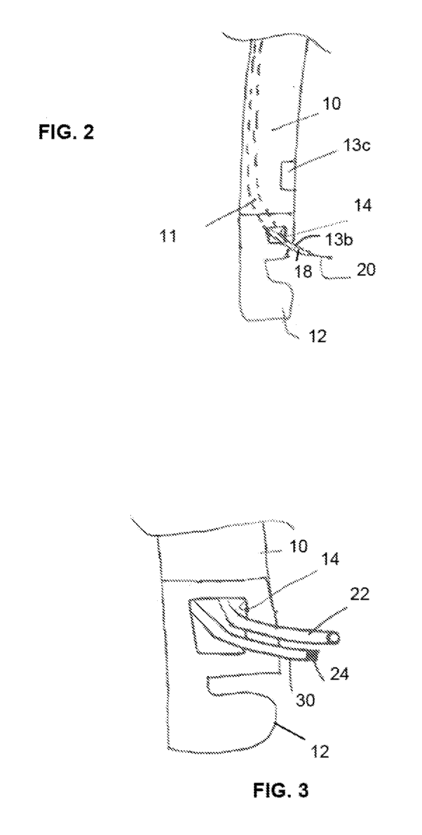

Referring to FIG. 2, the access device 10 includes a side port 14 in communication with an instrument channel 11 or lumen of the access device 10. A therapy device 16 includes a tubular body 18 and a first electrode in the form of a conductive needle tip 20. A lumen or channel within the tubular body 18 is in fluid communication with distal and/or sidewall openings in the needle tip 20 and/or in the sidewall of the tubular body proximal to the needle tip. A second, counter, electrode cooperates with the first electrode to create the electric field that will drive therapeutic substance ions into the surrounding tissue. The counter electrode maybe positioned in various locations. Examples of counter electrodes include surface patch electrodes 13a (FIG. 1), an internal electrode 13b (FIG. 2) within or on the tubular body 18, an internal electrode 13c (FIG. 2) within or on the access device 10, or an electrode positioned on a separate catheter introduced transorally through the stomach, duodenum and ampulla of vater into the pancreatic duct or common bile duct, or other suitably positioned electrodes. The therapy device 16 is extendable through the instrument channel/lumen and out the side port 14 as shown. The system includes a source 42 of therapeutic agent in ionized form fluidly coupled to the tubular body 18 (e.g. a reservoir within the therapy device 16 or an external reservoir separately coupled to the tubular body), and an electric source 44 coupled to the first and second electrodes to generate the electric field.

To treat pancreatic cancer, the access device 10 is introduced into the stomach via the mouth and advanced distally. The needle electrode 20 is advanced under ultrasonic guidance into or proximate the target tissue (e.g. a tumor). For tumors in the head or neck of the pancreas, the needle electrode can be placed through the wall of the duodenum and into the pancreas. For tumors in the body or tail of the pancreas, the needle electrode may be passed through the wall of the stomach into the pancreas. The therapeutic substance in ionic form is administered through the lumen and out the distal openings of the therapy device 16 and is driven into the target tissue using the electric field generated by the electrodes 20, 13.

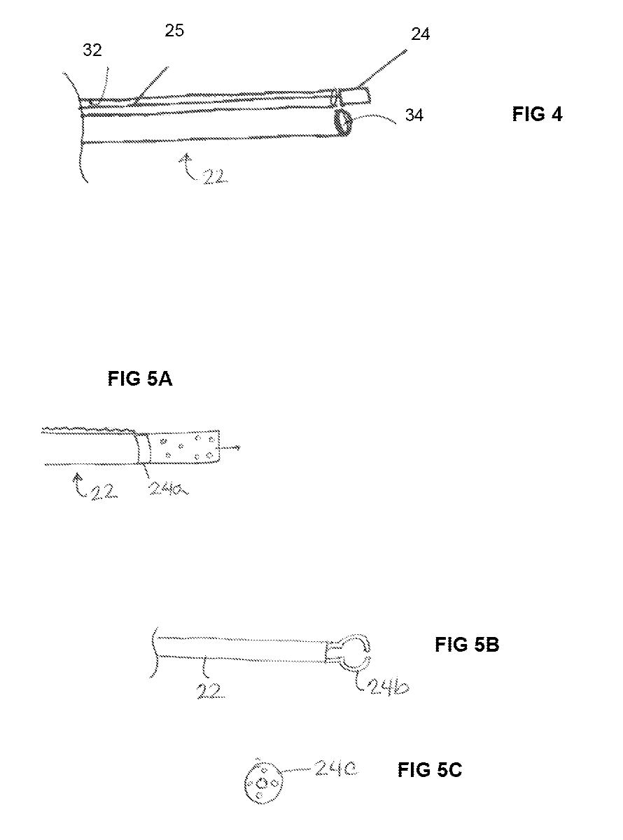

In a second embodiment, a delivery needle is used to facilitate positioning of an iontophoretic deliver catheter. The delivery needle may be similar to the needle shown in FIG. 2, although the channel through the needle and the conductive needle material may be eliminated. In particular, the delivery needle is advanced out of the side port 14 and into the target tissue in a manner similar to that described with respect to FIG. 1. A catheter 22 is advanced over the delivery needle into the target tissue, and the needle is then retracted and/or withdrawn from the body, leaving the catheter 22 in place. The catheter is used to deliver therapeutic substance into the body. If iontophoretic therapy is desired, two or more electrodes are positioned to create an electrical field that will aid in driving the therapeutic substance into the tissue. The first electrode may be positioned on a separate catheter 30 that is passed through the side port 14, and positioned in proximity to the catheter 22 employed to deliver the therapeutic substance. Alternatively, the catheter 22 may include a second channel/lumen 32 separate from its drug delivery channel 34 as shown in FIG. 4, allowing the conductor 25 of electrode element 24 to extend through the second channel and to be positioned in proximity to the exit port in the drug delivery channel. In yet another modification shown in FIG. 5, the drug delivery catheter 22 may be equipped with an electrode, such as a ring electrode 24a (FIG. 5A) on the catheter body. In alternative electrode designs, hoop-type electrodes 26 (FIG. 5B) may extend from the distal tip of the catheter 22, and/or a plate 26 may cover the distal end of the catheter 22 and including a plurality of openings for passage of the therapeutic substance as shown in FIG. 5C. The conductors for the electrodes may extend through lumens in the sidewalls of the catheter, or they may extend external to the catheter as shown.

As discussed above, in any of the disclosed embodiments, the second, counter, electrode (and any additional electrode(s)) may be positioned in any number of locations, including at a separate location on the catheter spaced apart from the first electrode, on a separate endoscopic instrument or catheter exiting the endoscopic access device 10 through the side port 14 or through a separate port, on the exterior of the access device 10, an a separate instrument positioned in a nearby blood vessel or duct (e.g. the pancreatic or biliary duct), and/or via surgical or percutaneous placement into or proximate the target tissue.

Alternative embodiments include placement of any of the above-described catheter systems (or variations thereof) using the following techniques: endoscopic placement into the pancreatic duct using endoscopic retrograde cholangiopancreatography (ERCP), percutaneous placement into or proximate target tissue (may use ultrasound or CT guidance), surgical (open or minimally-invasive) placement into or proximate target tissue, or interventional placement into or proximate the target tissue via a blood vessel (which may be exited to approach the target tissue).

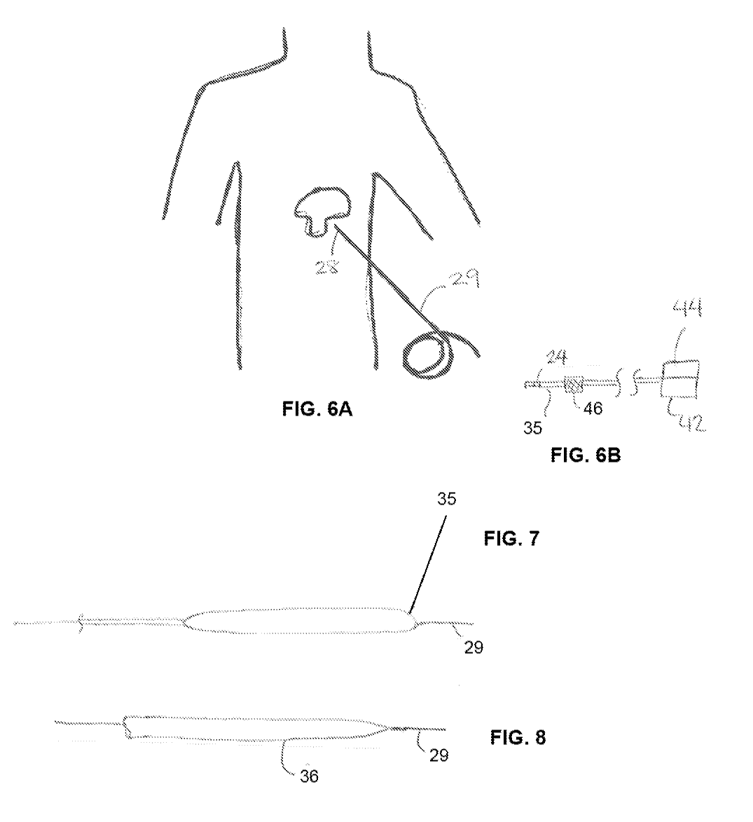

In a modification to the second embodiment, over-the-wire catheter placement may be employed for percutaneous positioning of a catheter. According to this modification, a hollow needle 28 disposed on a stiff guidewire 29 is inserted through the skin and placed at the target site using ultrasound or CT guidance as shown in FIG. 6. This procedure may be facilitated by the use of laparoscopic trocars or similar minimally invasive access devices.

The needle is disengaged from the guidewire (e.g. by sliding the hollow needle proximally over the guidewire), leaving the guidewire in place. If dilation surrounding the guidewire is needed to facilitate advancement of the therapeutic catheter, an over-the-wire balloon dilator 35 (FIG. 7) and/or stiff graduated dilator 36 (FIG. 8) is tracked over the guidewire so as to dilate the tissue surrounding the guide wire. The dilator is removed from the guidewire and a flexible therapeutic catheter 38 is then advanced over the guidewire to the target site. An anchor 46 is deployed to anchor the catheter at the target site, and the guidewire is withdrawn leaving the catheter anchored within the body. The anchor may take a variety of forms, including that of an expandable anchor having a stent-like configuration or an inflatable cuff,

The system includes two or more electrodes, one or both of which may be mounted to the catheter (see electrode 24) and/or mounted to a conductor extendable through a channel in the catheter. In either case, the electrodes are electrically coupled to a power supply 44 as described above. A channel in the catheter is fluidly coupled to a source 42 of ionized therapeutic substance. The catheter may be part of a system that includes a controller programmed to control the energy delivery and the dosing of the therapeutic substance.

While disclosed with reference to use for iontophoretic delivery of therapeutic substances, the disclosed embodiments may be adapted for use for non-iontophoretic drug delivery and/or for therapeutic energy delivery.

All prior patents and patent applications referred to herein, including for purposes of priority, are incorporated herein by reference.

I claim:

1. A method of delivering a therapeutic agent to pancreatic tissue, comprising:
transorally introducing into the gastro-intestinal tract the distal end of an endoscopic device;
advancing a delivery element from the endoscopic device through a wall of the gastro-intestinal tract and into an adjacent pancreas;
emitting a therapeutic agent from the delivery element into the pancreas; and
generating an electric field to drive the emitted therapeutic agent into surrounding tissue of the pancreas, causing the emitted agent to penetrate the surrounding tissue.

2. The method of claim 1, wherein the delivery element includes a first electrode, and wherein the method further includes generating the electric field between the first electrode and a second electrode.

3. The method of claim 2, wherein the second electrode is positioned on the delivery element.

4. The method of claim 2, wherein the second electrode is positioned on the endoscopic device.

5. The method of claim 2 wherein the second element is positioned on the skin of the subject.

6. The method of claim 2, wherein the method includes advancing a second element from the endoscopic device, wherein the second electrode is positioned on the second element.

7. The method of claim 2, wherein the first electrode comprises a conductive needle, and wherein advancing the delivery element through a wall of the gastro-intestinal tract comprises piercing the wall using the needle.

8. The method of claim 1, wherein the wall is a stomach wall.

9. The method of claim 1, wherein the wall is an intestinal wall.

10. A method of delivering a therapeutic agent to diseased tissue, comprising:
passing a catheter through an opening formed through body tissue and into the body;
positioning a drug delivery port of the catheter in proximity to target tissue to be treated;
emitting a therapeutic agent from the drug delivery port into the body; and
generating an electric field to drive the emitted therapeutic agent into the target tissue, causing the emitted agent to penetrate the target tissue.

11. The method of claim 10, wherein the catheter includes a first electrode, and wherein the method further includes generating the electric field between the first electrode and a second electrode.

12. The method of claim 11, wherein the second electrode is positioned on the catheter.

13. The method of claim 11 wherein the second element is positioned on the skin of the subject.

14. The method of claim 10, further including the step of expanding an anchor on the catheter to retain the drug delivery port in proximity to the target tissue.

15. The method of claim 10, wherein passing the catheter includes penetrating the body tissue using a needle having a guidewire coupled thereto, withdrawing the needle leaving the guidewire extending through the body tissue, and advancing the catheter over the guidewire into the body.
